(19) United States
(12) Patent Application Publication
(10) Pub. No.: US 2023/0222943 A1
(43) Pub. Date: Jul. 13, 2023

(54) TACTILE SYNTHETIC BONES

(71) Applicant: TACTILE ORTHOPAEDICS INC., Calgary (CA)

(72) Inventors: Gwendolyn Mary-jean Morgan, Calgary (CA); Jacqueline Claire Moreland, Calgary (CA); Carolyn Ruth Anglin, Calgary (CA); Christopher Ryan Martin, Calgary (CA)

(21) Appl. No.: 17/928,178

(22) PCT Filed: May 28, 2021
(86) PCT No.: PCT/CA2021/050729

§ 371 Date: Nov. 28, 2022

Related U.S. Application Data
(60) Provisional application No. 63/031,766, filed on May 29, 2020.

(51) Int. Cl. G09B 23/30

(52) U.S. Cl. CPC G09B 23/30

(57) ABSTRACT

The present disclosure relates to synthetic bones used in bone-related human and animal education, product demonstration, product development, surgical technique discussions, anatomical demonstrations and biomechanical research. The synthetic bones consisting of cortical an cancellous bones containing a plastic casting resin, a pore inducing additive and a hardness-altering additive.

### CROSS REFERENCE TO RELATED APPLICATION

This application claims priority to U.S. Provisional Patent Application No. 63/031,766, filed May 29, 2020, the entire contents of which is hereby incorporate by reference.

### FIELD

The present disclosure relates generally to synthetic bones. In some examples, the synthetic bones may be used in bone-related human or animal medical education, product demonstrations, product development, surgical technique discussions, anatomical demonstrations, and biomechanical research.

### BACKGROUND

There is increasing recognition that training outside the operating room (OR) benefits the learner, the patient and the healthcare system.

Compared to operating on patients, a learner using synthetic bones in a simulated setting, who may be a surgical trainee (e.g., a resident) or an experienced surgeon learning new tools or techniques, may have a more focused learning experience, can safely make mistakes (even deliberately making mistakes to understand the outcome), can complete the entire procedure rather than just a small part, and has the opportunity to improve knowledge, skills and confidence.

Patients benefit by not necessarily being the first experience of the surgeon with a new procedure, and may have a higher likelihood of a good outcome.

The healthcare system may benefit by having fewer complications (which result in more follow-up visits and possible revision surgery) and shorter surgeries.

Put another way, it is preferred to practice on a non-patient, before practicing on a patient.

Surgeries are hands-on procedures in which the tactile feedback plays an important role. Replicating the transitional feel of harder cortical bone to softer cancellous bone is essential to allow a model to match the tactile surgical environment. Surgeons have no objective method of judging how much force they should use when performing tasks that remove bone. Therefore, they rely on feel. Common orthopaedic tasks that involve removing bone include drilling and sawing.

Matching the material properties of bone is not enough. Accurate material properties need to be combined with accurate bone geometry because the feel of bone changes based on the location of bone being drilled, angle of drilling and depth of drilling.

The transitional feel of the harder cortical bone to softer cancellous bone is important to teach surgeons how to appropriately adjust their force of drilling. Inappropriate drilling often results in plunging, which can damage sensitive structures. This is frequently seen with junior residents. Having accurate differential bone also helps teach the feel of drilling bone tunnels at different angles for ligament reconstruction surgery.

In knee joint replacement, instrumentation or implants are often placed into the medullary canal. The material in the canal is even softer than the cancellous material at the ends of the bones.

The differential bone feel between cortical and cancellous bone is different based on the patient population the model is simulating. Arthroscopic ligament surgery is preferentially done on younger active patients with better cancellous bone quality. Knee replacement surgery (total knee arthroplasty) is preferentially done on older patients with arthritis. Different bone materials are described in this application that replicate a young active patient's cortical and cancellous bone, and for a different material, cortical and cancellous bone of an older patient.

The feel of the cortical and cancellous bone is also important to teach the tactile feel of different ligament fixation methods. For example, interference screws are used to secure a ligament/tendon graft in a tunnel by squeeze fit. Too tight a fit and the graft can be damaged during placement of the screw whereas too loose and the graft can slide within the tunnel because it is not fixed strongly enough. The tunnel angle is very important because it changes the size of the tunnel's aperture and subsequent graft fit. Accurately matching the bone feel can teach residents and surgeons proper technique on placement of interference screws.

In general terms, the question is: how hard or how softly should the learner press on the instruments? How should they properly hold the instruments? These are subtle differences that must be learned to maximize patient safety and outcome.

The better the tactile feel of the bone matches the normal experience with the patient, the less the psychological barrier is broken from the clinical to the simulated environment, and therefore the more immersed the learner can be in the training. They can feel as if they are in the operating room.

Fatty deposits exist in bone. Including simulated fatty deposits therefore adds further to the realism and immersion in the surgical experience.

Interactions between the instruments and the models may include: sawing, drilling, reaming, probing, inserting pins or screws, burring, hammering and so on.

Surgical simulation models generally fall into three main categories: (1) synthetic bones; (2) cadaveric specimens and (3) virtual reality.

(1) Current synthetic bones provide the basic shapes of the bones, but do not have a realistic tactile feel. There is a difference between the outer cortical and inner cancellous bone, but once into the inside bone there is limited resistance. Pins and screws can toggle, and implants may not seat properly onto the bone, instead bouncing back, leaving a gap. The bone shapes are often well away from the anatomical norm. They are generally not considered valuable beyond learning the basic steps of the instrumentation.

(2) Cadaveric specimens are more realistic, and have been routinely used for surgical training, but can only be used in dedicated facilities, are highly variable from specimen to specimen (thus creating unequal and unpredictable experiences), require special handling as well as preparation, cleanup and special disposal, are typically older people (thus not representing younger patients), are biohazardous, and are clearly unsuitable for remote learning.

(3) Virtual reality is useful as a first introduction to a procedure, and allows for repeated practice, but is still not able to fully replicate the tactile feel and forces of the instruments and the relationship to the patient anatomy or positioning. Having a physical model allows the real instruments and implants to be used, and muscle memory to be trained and practiced.

A robust synthetic bone model with a realistic tactile feel that is not biohazardous provides new opportunities for more advanced training anywhere.

### SUMMARY

In one aspect there is provided a synthetic cortical bone comprising:
a plastic casting resin, and
a pore-inducing additive.

In one aspect there is provided a synthetic cortical bone comprising:
a plastic casting resin,
a hardness-altering additive, and
a pore-inducing additive.

In one example, further comprising a fiber.

In one example, further comprising a tint.

In one example, wherein said plastic casting resin is Smooth-Cast 300, Smooth-Cast 321, MirrorCast, Smooth-Cast 385, Smooth-Cast 380, Smooth-Cast ONYX, Task 15, Task 2, Task 11 or Smooth-Cast 60D.

In one example, said plastic casting resin is Smooth-Cast 300.

In one example, said casting resin is Task 2 or Task 11.

In one example, said plastic casting resin in Task 2.

In one example, said pore-inducing agent is bentonite, wood ash, fly ash, volcanic ash, soda ash, kaolinite, talc, montmorillonite or calcium carbonate.

In one example, said pore-inducing agent is bentonite.

In one example, comprising about 1% to about 50%, preferably about 1% to about 25% bentonite, preferably about 25% to about 50% bentonite, preferably about 30% to about 35% bentonite, relative to the total amount of plastic resin.

In one example, said pore-inducing agent is calcium carbonate.

In one example, comprising about 1% to about 10% calcium carbonate, relative to the total amount of plastic resin.

In one example, said hardness-altering agent is collagen, AquaResin powder, Goldfinger Powder, Ure-Fil 7, QuarryTone, Ure-Fil 3, Ure-Fil 15, gelatin, Ure-Fil 11, calcium carbonate, or wood flour.

In one example, the hardness-altering agent is collagen.

In one example, comprising from about 1% to about 50% collagen, preferably about 1% to about 10% collagen, preferably about 1% to about 33% collagen, relative to the total amount of plastic resin.

In one example, comprising about 1%, about 2%, about 3%, about 4%, about 5%, about 6%, about 7%, about 8%, about 9%, about 10% collagen, relative to the total amount of plastic resin.

In one example, wherein the hardness-altering agent is wood flour.

In one example, comprising from about 5% to about 15% wood flour, relative to the total amount of plastic resin.

In one example, comprising about 5%, about 6%, about 7%, about 8%, about 9%, about 10%, about 11%, about 12%, about 13%, about 14%, or about 15% wood flour, relative to the total amount of plastic resin.

In one example, wherein the fiber is hemp, jute, flax, ramie, sisal, bagasse, wood fibers, bamboo fibers, wood shavings, bleached or unbleached kraft, animal fibers, silk, wool, semi-synthetic fiber, nylon, Dacron, rayon, acrylic polyesters, cotton, tencel lyocell, linen (flax), or alkali resistant glass fibers.

In one example, the fiber is hemp.

In one example, comprising about 0.5% to about 3% hemp, relative to the total amount of plastic resin.

In one example, the tint is a powdered tint or So-Strong™.

In one example, the Shore hardness value is between about 60D to about 95D.

In one aspect there is provided a synthetic cancellous bone, comprising:
a plastic casting resin, and
a pore-inducing additive

In one aspect there is provided a synthetic cancellous bone, comprising:
a plastic casting resin,
a softening additive, and
a pore-inducing additive.

In one example, further comprising a tint.

In one example, the plastic casting resin is Task 2 or Task 11.

In one example, the softening additive is wood flour or collagen.

In one example, the pore-inducing agent is calcium carbonate, bentonite, wood ash, fly ash, volcanic ash, soda ash, kaolinite, talc or montmorillonite.

In one example, the pore-inducing agent additive is calcium carbonate.

In one example, comprising from about 1% to about 10% calcium carbonate, preferably about 2% to about 7% calcium carbonate, relative to the total amount of plastic resin.

In one example, said pore-inducing agent additive is bentonite.

In one example, comprising from about 5% to about 20% bentonite, relative to the total amount of plastic resin.

In one example, comprising about 10%, about 11%, about 12%, about 13%, about 14%, or about 15% bentonite, relative to the total amount of plastic resin.

In one example, comprising from about 5% to about 30% wood flour, preferably about 7% to about 22% wood flour, relative to the total amount of plastic resin.

In one example, comprising about 7%, about 8%, about 9% about 10%, about 11%, about 12%, about 13%, about 14%, about 15%, about 16%, about 17%, about 19%, about 20%, about 21%, or about 22% wood flour, relative to the total amount of plastic resin.

In one example, the tint is a powdered tint or So-Strong™.

In one example, the Shore hardness value is between about 35D to about 85D.

In one aspect there is provided a synthetic bone comprising:

an inner synthetic cancellous bone core, comprising the synthetic cancellous bone of any one of claims 26 to 39; and

an outer synthetic cortical bone layer, comprising the synthetic cortical bone of any one of claims 1 to 25.

In one aspect there is provided a synthetic cancellous bone comprising:

an inner synthetic cancellous bone core, comprising the synthetic cancellous bone of any one of claims 26 to 41; and

a fat-simulating substance.

In one example, the fat-simulating substance is petroleum jelly, glycerin, soft paraffin and/or mineral oil.

In one example, the fat-simulating substance is petroleum jelly, preferably about 1% to about 20%.

In one aspect there is provided a kit for a synthetic cancellous bone, comprising: the synthetic cancellous bone of any one of claims 26 to 44, and a container, and optionally instructions for use.

In one aspect there is provided a kit for a synthetic cortical bone, comprising the synthetic cortical bone of any one of claims 1 to 25, and a container, and optionally instructions for use.

In one aspect there is provided a kit for a synthetic bone, comprising:

an inner synthetic cancellous bone core, comprising the synthetic cancellous bone of any one of claims 26 to 44;

an outer synthetic cortical bone layer, comprising the synthetic cortical bone of any one of claims 1 to 25; and a container, and optionally instructions for use.

In one aspect there is provided a kit for a synthetic bone block, comprising:

a cortical-cancellous or cortical-cancellous-cortical construct in a non-bone-like shape, comprising the synthetic cortical bone of any one of claims 1 to 25; the synthetic cancellous bone of any one of claims 26 to 44; a container, and optionally instructions for use.

### BRIEF DESCRIPTION OF THE DRAWING

Embodiments of the present disclosure will now be described, by way of example only, with reference to the attached Figures.

FIG. 1A depicts an example of a Tactile Knee™ model, shown with a skin sleeve covering the knee.

FIG. 1B depicts an example of an arthroscopy Tactile Knee™ model, replicating younger bone.

FIG. 1 C depicts an example of an arthroplasty Tactile Knee™ model, replicating older bone, including arthritis.

FIG. 2A depicts a synthetic femur bone (bottom portion).

FIG. 2B depicts a synthetic tibia bone (top portion).

FIG. 3A depicts a cross-section through the tibia, showing the outer cortical and inner cancellous bone for the younger bone

FIG. 3B depicts a cross-section through the tibia, showing the outer cortical and inner cancellous bone for the older bone.

FIG. 4 depicts an X-ray of the full knee model showing realistic radiodensity.

### DETAILED DESCRIPTION

Generally, the present disclosure provides for synthetic bones.

As used herein, the term “synthetic bone” refers to a device and/or material. In some examples, “synthetic bone” may also be referred to as an “artificial bone” or “bone analogue”, which refers to a bone-like material in a state that is different from a natural state.

Described herein are synthetic bones that balance being hard enough to feel like bone while being ductile enough that they do not fracture, and also avoid a “plasticky” feel and look when drilling into the synthetic bone.

Generally, a mammalian bone comprises an inner core (cancellous bone) and an outer layer (cortical bone).

Mammals include but are not limited to domesticated animals, such as cats, dogs, etc., livestock (e.g., cattle, horses, pigs, sheep, goats, etc.), laboratory animals (e.g., mouse, rabbit, rat, guinea pig, etc.), non-human mammals, primates, non-human primates, rodents, and any other animal. In a specific example, the mammal is a human.

In accordance with one aspect of the present disclosure, one or more formulations of a synthetic cortical bone is provided.

In accordance with one aspect of the present disclosure, one or more formulations of a synthetic cancellous bone is provided.

In accordance with another aspect of the present invention, a synthetic bone is provided comprising an inner synthetic cancellous bone core and an outer synthetic cortical bone layer.

Synthetic Cortical Bone

In one example, there is provided a synthetic cortical bone comprising:
a plastic casting resin, and
a pore-inducing additive.

In one example, there is provided a synthetic cortical bone comprising:
a plastic casting resin,
a hardness-altering additive, and
a pore-inducing additive.

Optionally, the synthetic cortical bone further comprises a fiber.

Optionally, the synthetic cortical bone further comprises a tint.

In some examples, the synthetic cortical bone has a Shore hardness of between about 60D to about 100D. In one example, the synthetic cortical bone has a Shore hardness of about 80D to about 100D. In one example, the synthetic cortical bone has a Shore hardness of about 60D to about 85D. In one example the synthetic cortical bone has a Shore hardness of about 85D to about 95D.

In one example, the synthetic cortical bone has a Shore hardness of about 60D, 61D, 62D, 63D, 64D, 65D, 66D, 67D, 68D, 69D, 70D, 71D, 72D, 73D, 74D, 75D, 76D, 77D, 78D, 79D, 80D, 81D, 82D, 83D, 84D, 85D, 86D, 87D, 88D, 89D, 90D, 91D, 92D, 93D, 94D, 95D, 96D, 97D, 98D, 99D, or 100D.

In some examples, the properties of the synthetic cortical bone may include one or more of the following: has the tactile feel of cortical bone (for example as judged by a surgeon); is machinable, is non-toxic, does not fracture under normal loads or separate from the cancellous during drilling or sawing; does not have a distinctive odor, debris does not spiral (as with normal plastic), has realistic X-ray radiodensity, has a statistically-average shape for anatomical accuracy, is dimensionally consistent, with different bone geometries possible, similar thickness of cortical layer to human bone, may be used to simulate human or animal bone, has a more realistic bone-like texture compared to plastic resin on its own, is waterproof (which is useful for certain surgical procedures), can be easily adhered to, is minimally responsive to environmental factors (temperature, humidity etc); and preferably has a short cure time and/or adheres to cancellous.

In some examples, a plastic casting resin is water resistant in the cured state, and/or odorless or substantially odorless. In some examples, a plastic casting resin provides strength, low cure times, and/or high resolution.

Examples of a “plastic casting resin” include, but are not limited to, Smooth-Cast 300, Smooth-Cast 321, MirrorCast, Smooth-Cast 385, Smooth-Cast 380, Smooth-Cast ONYX, Task 15, Task 2, Task 11, Smooth-Cast 60D, or said plastic casting resin is Smooth-Cast 321.

In one example, in the case of synthetic cortical bones that simulate older bone Task 2 or Task 11 may be used.

In another example, said plastic casting resin is Task 2 or Task 11.

In one example, said plastic casting resin is Task 2.

In one example, said pore-inducing agent is bentonite, wood ash, fly ash, volcanic ash, soda ash, kaolinite, talc, montmorillonite, or calcium carbonate.

In one example, said pore-inducing agent is bentonite, preferably about 1 to 50% bentonite, relative to the total amount of plastic resin. In some examples, the pore-inducing agent is bentonite, preferably about 35% bentonite, relative to the total amount of plastic resin.

In one example, said pore-inducing agent is calcium carbonate, preferably about 1% to about 10% calcium carbonate, relative to the total amount of plastic resin, or preferably about 1%, about 2%, about 3%, about 4%, about 5%, about 6%, about 7%, about 8%, about 9%, or about 10%, calcium carbonate relative to the total amount of plastic resin.

In some examples, Smooth-Cast refers to a fast-curing, machinable, non-toxic, 2-part liquid casting plastic to address fracture toughness, and/or is easier for adhesion than a plaster-based formulation (Smooth-On, Inc; https://www.smooth-on.com/products/smooth-cast-300/).

In a specific example of a plastic casting resin, Smooth-Cast 300 is used. Smooth-Cast 300 is a casting compound consisting of two liquid prepolymers that can be mixed and cured to form a durable plastic.

Examples of a “hardness-altering agent” include but are not limited to collagen, AquaResin powder, Goldfinger Powder, Ure-Fil 7, QuarryTone, Ure-Fil 3, Ure-Fil 15, gelatin, Ure-Fil 11, or wood flour. In a specific example, collagen is used.

In one example, the hardness-altering agent is collagen. Preferably about 1% to about 50%, preferably about 1% to about 10% collagen, relative to the total amount of plastic resin. More preferably, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, relative to the total amount of plastic resin.

In one example, the hardness-altering agent is wood flour. Preferably from about 5% to about 15% wood flour, relative to the total amount of plastic resin. More preferably about 5%, about 6%, about 7%, about 8%, about 9%, about 10%, about 11%, about 12%, about 13%, about 14%, or about 15% wood flour, relative to the total amount of plastic resin.

The term “pore-inducing” may also be referred to as “density reducing”. Examples of “pore-inducing additive” include but are not limited to bentonite, wood ash, fly ash, volcanic ash, soda ash, kaolinite, talc montmorillonite, calcium carbonate, or bentonite.

Bentonite was found to provide a different feel than collagen.

In one example, said pore-inducing agent wherein said pore-inducing agent is bentonite. In some examples, the bentonite comprising about 1% to about 50%, preferably about 1% to about 25% bentonite, preferably about 25% to about 50% bentonite, preferably about 30% to about 35% bentonite, relative to the total amount of plastic resin. In some examples the bentonite comprising about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% bentonite, relative to the total amount of plastic resin.

In one example, said pore-inducing agent is calcium carbonate.

Preferably about 1% to about 10% calcium carbonate, relative to the total amount of plastic resin. More preferably, about 1% to about 10% calcium carbonate, relative to the total amount of plastic resin.

Examples of a “fiber” include but are not limited to a natural or synthetic substance that is significantly longer than it is wide. As used herein, “fiber component” means a source of fiber or fibers, which fibers may be natural fibers (vegetable, wood and animal) or semi-synthetic or synthetic fibers. Fiber component may comprise vegetable fibers, which are generally based on an arrangement of cellulose and lignin, including cotton, hemp, jute, flax, ramie, sisal, bagasse and banana; wood fibers, from tree sources, which include bamboo fibers, wood shavings, bleached or unbleached kraft or sulfite pulps that are used for making paper; animal fibers such as silk and wool; semi-synthetic fibers such as nylon, Dacron and rayon; and synthetic fiber such as acrylic polyesters and alkali resistant glass fibers. In a specific example, the fiber is hemp. In another specific example the fiber is pulp and paper. In some examples, the fibers break up the debris while drilling, resulting in more separate particles (more powdery) rather than typical plastic spirals; it also helps with the strength.

Examples of a “tint” (which may also be referred to as a “coloring agent”) include, but are not limited to, powdered tints of differing color (e.g., yellow, red, brown, or blends of color). In another example, a tint includes, but is not limited to, So-Strong™ from Smooth-on (https://www.smooth-on.com/product-line/strong/).

In one example, from 1% to 50% collagen may be used, relative to the total amount of the plastic resin (parts A & B). In another example, from 5% to 7% collagen may be used, relative to the total amount of the plastic resin (parts A & B)

In one example, from 1% to 50% bentonite may be used, relative to the total amount of the plastic resin. In another example, from 30% to 35% bentonite may be used, relative to the amount of the plastic resin.

In one example, from 0.5% to 3% hemp may be used, relative to the total amount of the plastic resin. In another example, from 1% to 1.5% hemp may be used, relative to the total amount of the plastic resin.

Synthetic Cancellous Bone

In one example, there is provided a synthetic cancellous bone, comprising:
a plastic casting resin, and
a pore-inducing additive.

In one example, there is provided a synthetic cancellous bone, comprising:
a plastic casting resin,
a softening additive, and
a pore-inducing additive.

Optionally, the synthetic cancellous bone further comprises a tint.

In some examples, the synthetic cancellous bone has a Shore hardness of between about 35D to 80D.

The properties of the synthetic cancellous bone may include one or more of the following: tactile feel of cancellous with surgical instruments (for example, as judged by a surgeon), tactile feel of cortical-cancellous transition (for example, as judged by a surgeon), realistic porosity (for example, as judged by a surgeon), realistic pin purchase/implant seating (vs toggling, or leaving a gap), realistic X-ray radiodensity, range of mechanical properties or densities possible (i.e. younger vs older bone), realistic inhomogeneity/porosity, waterproof, may simulate human or animal bone, structurally stable (e.g., can cut off slices of the tibia when doing knee joint replacement and they stay intact), is machinable, is non-toxic, the materials are readily available, or have short cure-time.

Examples of a cancellous plastic casting resin include, but are not limited to, Task 2 or Task 11. In a specific example, Task 2 is used.

Task 2 is a quick-setting, machinable, non-toxic 2-part flexible plastic casting resin (i.e. softer than cortical to ensure a cortical-cancellous transition); easy to manufacture, together with additives. Task 2 has a good tactile feel.

Examples of a cancellous softening additive include, but are not limited to, wood flour, or collagen. In some examples, wood flour was used to make the Task 2 softer.

Examples of cancellous pore-inducing additives include, but are not limited to, calcium carbonate, bentonite, wood ash, fly ash, volcanic ash, soda ash, kaolinite, talc or montmorillonite. In some examples, calcium carbonate created porosity by turning the cancellous plastic casting resin and wood flour mixture into a foam.

In one example, the pore-inducing agent additive is calcium carbonate. Preferably from about 1% to about 10% calcium carbonate, relative to the total amount of plastic resin.

In one example, said pore-inducing agent additive is bentonite. Preferably from about 10% to about 15% bentonite, relative to the total amount of plastic resin. More preferably about 10%, about 11%, about 12%, about 13%, about 14%, or about 15% bentonite, relative to the total amount of plastic resin.

In one example, the softening additive comprising from about 7% to about 22% wood flour, relative to the total amount of plastic resin. Preferably about 7%, about 8%, about 9% about 10%, about 11%, about 12%, about 13%, about 14%, about 15%, about 16%, about 17%, about 19%, about 20%, about 21%, or about 22% wood flour, relative to the total amount of plastic resin.

Examples of “tints” (which may also be referred to as a “coloring agent”) include, but are not limited to, powdered tints of differing color (e.g., yellow, red, brown, or blends of color). In another example, a tint includes, but is not limited to, So-Strong™ from Smooth-on (https://www.smooth-on.com/product-line/strong/).

In one example, from 8% to 20% wood flour may be used, relative to the total amount of plastic resin (parts A and B).

In one example, from 2% to 5% calcium carbonate may be used, relative to the total amount of plastic resin.

Synthetic Bone Comprising an Inner Synthetic Cancellous Bone Core and an Outer Synthetic Cortical Bone Layer

In one example, there is provided a synthetic bone comprising:
an outer synthetic cortical bone, and
an inner synthetic cancellous bone.

In one example, there is the outer synthetic cortical bone comprising:
a plastic casting resin, and
a pore-inducing additive.

In one example, there is provided the outer synthetic cortical bone comprising:
a plastic casting resin,
a hardening additive, and
a pore-inducing additive.

Optionally, the outer synthetic cortical bone further comprises a fiber.

Optionally, the outer synthetic cortical bone further comprises a tint.

In one example, the inner synthetic cancellous bone, comprises:
a cancellous plastic casting resin, and
a cancellous pore-inducing additive

In one example, the inner synthetic cancellous bone, comprises:
a cancellous plastic casting resin,
a cancellous softening additive, and
a cancellous pore-inducing additive.

Optionally, the inner synthetic cancellous bone further comprises a tint.

In some examples, the synthetic bone or synthetic cancellous bone further comprises a fat-simulating substance. In one example, the fat-simulating substance is petroleum jelly, glycerin, soft paraffin and/or mineral oil. In a specific example, the fat-simulating substance is petroleum jelly.

Manufacture of Synthetic Cortical Bone and Synthetic Cancellous Bone and Synthetic Bone Comprising an Outer Synthetic Cortical Bone, and an Inner Synthetic Cancellous Bone.

In some examples, manual injection is used in the manufacture of the synthetic cortical bones described herein.

In some examples, pouring is used in the manufacture of synthetic cancellous bones.

In some examples, the inner synthetic cancellous bone is produced first, and then placed in a repeatable position within the outer synthetic cortical mold and the inner synthetic cortical material injected to surround the cancellous bone.

In another example, blocks of material may be created, whereby the outer synthetic cortical bone material may be molded as a hollow structure or a single sheet and the inner synthetic cancellous bone material pressed into, onto or injected inside the structure, and layered to simulate the tactile feel of the various bony structures.

In some examples, a conductive element such as conductive ink, silver, metal, carbon or activated charcoal may be added to either the inner core or the outer layer or both so that the inner core and/or the outer layer have different conductivities, as in real bone.

Uses

The synthetic bones described herein may be optionally combined with simulated muscle, fat, skin, ligaments, tendons, meniscus or cartilage to give the end-user a more realistic training system to practice on.

It will be appreciated that synthetic bones described herein may be utilized by one or more of the following end-users in human or veterinary medicine applications: medical students; medical residents (e.g., practicing arthroscopic surgery, knee joint replacement, or pedicle screw placement); surgeons (e.g., navigation techniques, certification, re-certification, practicing a case preoperatively on a patient-specific generated model, training residents, or demonstrating the anatomy to a patient); dentist trainees (e.g., practicing dental drilling); engineers or technicians (e.g., conducting product verification testing or biomechanical testing); sales personnel (e.g., product demonstrations); educators (e.g. anatomical teaching to students and patients); and children (e.g., educational toys).

In some examples, the synthetic bones described herein may include, but are not limited to, femur, tibia, patella, fibula, spine, pelvis, humerus, scapula, clavicle, ulna, radius, foot, hand, hip, knee, ankle, shoulder, wrist, leg, arm, mandible, maxilla, sacrum, skull, sternum, ribs, teeth, bone marrow, encompassing all bones and joints of the body.

The synthetic bones described herein may be used for conducting one or more of the following procedures or any other bone-related procedure: arthroscopic techniques, joint replacement surgery (e.g. knee, hip, and shoulder), surgical navigation techniques (e.g. for joint replacement), fracture fixation (e.g. femur, tibia, humerus, wrist, and ankle), pedicle screw placement, dental drilling, injection, targeting-sawing-drilling and repairing.

The synthetic bones described herein may be used for conducting one or more of the following biomechanical tests or any other bone-related biomechanical testing: screw pull out, orthopaedic implant testing and orthopaedic instrument testing.

In some examples, the synthetic bones described herein may be used for product demonstrations that use bone models to illustrate aspects of the product.

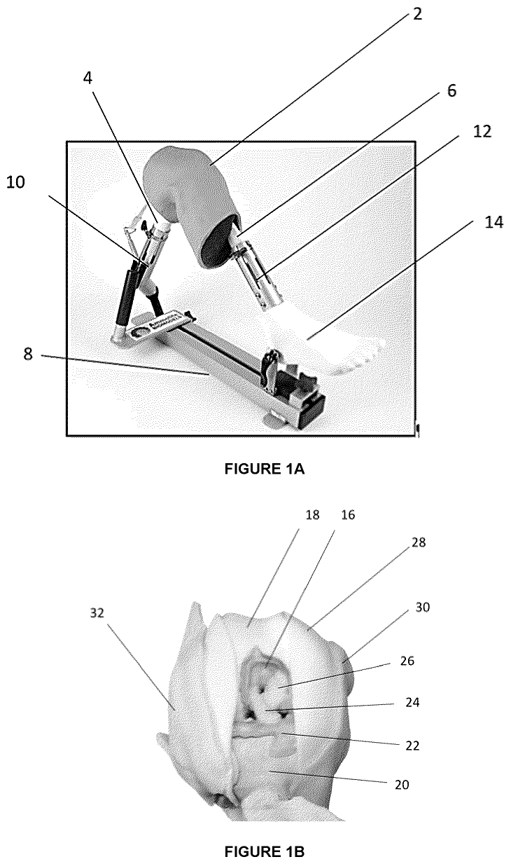

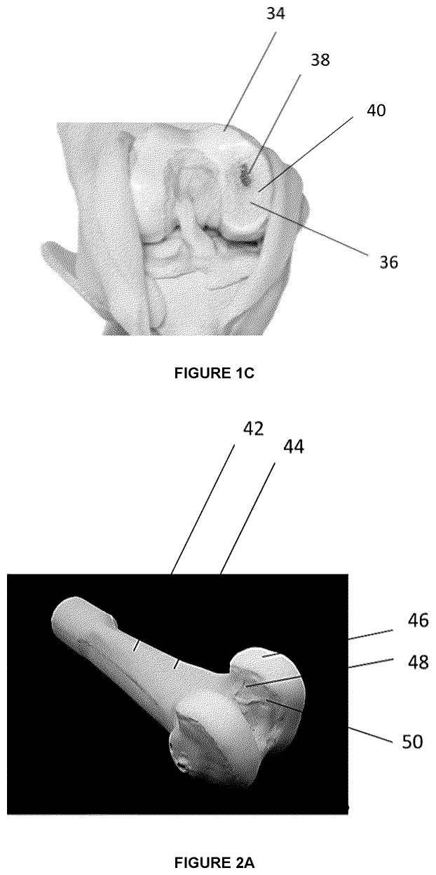

FIG. 1A depicts an example of a Tactile Knee™ model, shown with a skin sleeve covering the knee, held in the dynamic knee positioner. In FIG. 1A, the skin sleeve (2) covers the synthetic knee (not shown), which includes a femur (4) and tibia (6). The model is held in a dynamic knee positioner (8), which includes a femoral post (10), tibial post (12) and foot (14).

FIG. 1B depicts an example of an arthroscopy Tactile Knee™ model, replicating younger bone. In FIG. 1B, the femur (16), which is covered by cartilage (18), and the tibia (20), which is covered by cartilage (not shown) and menisci (22), are joined by the anterior cruciate ligament (ACL) (24), posterior cruciate ligament (PCL) (26), and joint capsule (28), including the medial collateral ligament (MCL) (30) and lateral collateral ligament (LCL) (32).

FIG. 1C depicts an example of an arthroplasty Tactile Knee™ model, replicating older bone, including arthritis. In FIG. 1C, the femoral cartilage (34) is depicted worn away, replicating medial osteoarthritis (36), revealing the cancellous bone (38) below the cortical bone (40).

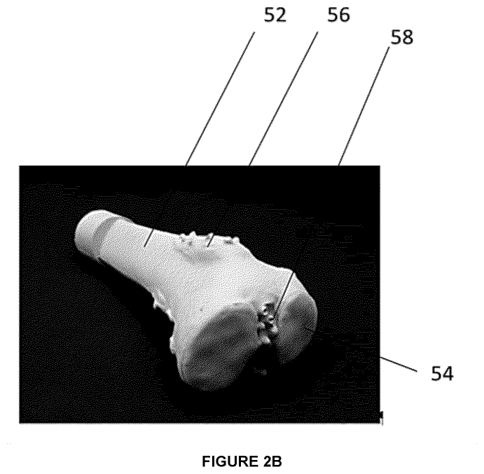

FIG. 2A depicts a synthetic femur bone (bottom portion). In FIG. 2A, the distal part of the femur (42) is depicted with the outer cortical bone (44) and inner cancellous bone (not shown), with the condyles (46) covered in cartilage. Specific anatomic landmarks, the bifurcate ridge (48) and intercondylar ridge (50), are depicted on the femur (42).

FIG. 2B depicts a synthetic tibia bone (top portion). In FIG. 2B, the proximal part of the tibia (52) is shown, with cartilage (54) covering the tibial plateau, as well as attachment points (56) for the patellar ligament and attachment points for the ACL (58).

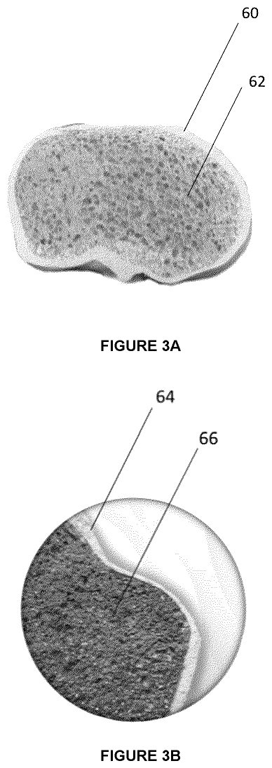

FIG. 3A depicts a cross-section through the tibia, showing the outer cortical bone (60) and inner cancellous bone (62) for the younger bone, suitable for the arthroscopy model.

FIG. 3B depicts a cross-section through the tibia, showing the outer cortical bone (64) and inner cancellous bone (66) for the older bone, suitable for the arthroplasty model.

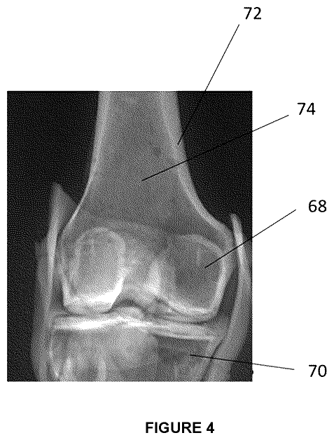

FIG. 4 depicts an X-ray of the full knee model showing realistic radiodensity of the femur (68) and tibia (70), including the outer cortical bone (72) and inner cancellous bone (74).

Methods of the invention are conveniently practiced by providing the compounds and/or compositions used in such methods in the form of a kit. Such a kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in any way.

### EXAMPLES

### Example 1—Synthetic Cortical Bone

Specific examples of a synthetic cortical bone are presented in Table 1 (younger bone) and Table 2 (older bone).

TABLE 1
Synthetic cortical bone formulations - for younger
bone (e.g. 20-30 years old); percentages are relative
to the total of the casting resin (parts A and B)

| | Amount | Amount | Amount |
|---|---|---|---|
| | for femur, | for tibia | for femur |
| Material | Example A (g) | Example A (g) | Example B (g) |
| Smooth-Cast | 73.9 | 62.2 | 66.6 |
| 300 - Part A | | | |
| Smooth-Cast | 66.6 | 56.0 | 60.0 |
| 300 - Part B | | | |
| Collagen | 7.2 (5%) | 6.1 (5%) | 42.2 (33%) |
| Bentonite | 46.8 (33%) | 39.4 (33%) | 6.4 (5%) |
| Hemp | 1.8 (1.3%) | 1.4 (1.3%) | N/A |
| Tint | 6.6 (5%) | 4.8 (5%) | 2.5 (2%) |
| Manufacturing | Inject with | Inject with | Vacuum Part B with |
| | syringe due | syringe due | all dry ingredients to |
| | to fibers | to fibers | de-gas; flows easily |
| | | | due to lack of fibers |
| Comment | Feels like | Feels like | Feels like cortical; |
| | cortical; | cortical; | debris spirals more; |
| | good debris | good debris | easier to manufacture |

TABLE 2
Synthetic cortical bone formulations - for older bone
(e.g. 50-60 years old); percentages are relative to
the total of the casting resin (parts A and B)

| | Amount | Amount |
|---|---|---|
| | for femur, | for tibia |
| Material | Example C (g) | Example C (g) |
| Smooth-Cast Task 2 - | 50.8 | 52.8 |
| Part A | | |
| Smooth-Cast Task 2 - | 46.0 | 46.0 |
| Part B | | |
| Wood Flour | 9.6 (10%) | 9.6 (10%) |
| Calcium Carbonate | 2.8 (3%) | 2.8 (3%) |
| Tint | 2 drops | 2 drops |
| Manufacturing | Poured into mould | Poured into mould |
| Comment | Softer than | Softer than |
| | examples A&B | examples A&B |

Specific examples of a synthetic cancellous bone are presented in Table 3 (younger bone) and Table 4 (older bone).

TABLE 3
Synthetic cancellous bone formulation - for younger
bone (e.g. 20-30 years old); percentages are relative
to the total of the casting resin (parts A and B)

| | Amount | Amount |
|---|---|---|
| Material | for femur (g) | for tibia (g) |
| Task 2 - Part A | 25.4 | 19.7 |
| Task 2 - Part B | 23.0 | 17.9 |
| Wood flour | 4.8 (10%) | 3.7 (10%) |
| Calcium carbonate | 1.4 (3%) | 1.1 (3%) |
| Tint | 1 drop | 1 drop |
| Manufacturing | Poured into mold | Poured into mold |
| Comment | Feels like young | Feels like young |
| | cancellous | cancellous |

TABLE 4
Synthetic cancellous bone formulation - for older bone
(e.g. 50-60 years old); percentages are relative to
the total of the casting resin (parts A and B)

| | Amount | Amount |
|---|---|---|
| Material | for femur (g) | for tibia (g) |
| Task 2 - Part A | 23.0 | 17.9 |
| Task 2 - Part B | 27.0 | 21.0 |
| Wood flour | 4.8 (10%) | 3.7 (10%) |
| Calcium carbonate | 1.4 (3%) | 1.1 (3%) |
| Bentonite | 6.5 (13%) | 5.0 (13%) |
| Tint | 2 drops | 2 drops |
| Manufacturing | Poured into mold | Poured into mold |
| Comment | Feels like older | Feels like older |
| | cancellous | cancellous |

TABLE 5
Synthetic cancellous bone formulation - for the
medullary canal; percentages are relative to
the total of the casting resin (parts A and B)

| | Amount | Amount |
|---|---|---|
| Material | for femur (g) | for tibia (g) |
| Task 2 - Part A | 17.9 | 17.9 |
| Task 2 - Part B | 21.0 | 21.0 |
| Wood flour | 7.4 (19%) | 7.4 (19%) |
| Calcium carbonate | 2.2 (6%) | 2.2 (6%) |
| Bentonite | 5.0 (13%) | 5.0 (13%) |
| Tint | 3.0 (8%) | 3.0 (8%) |
| Manufacturing | Poured into mold | Poured into mold |
| Comment | Softer than other | Softer than other |
| | formulations | formulations |

Sources of Materials

Smoothcast: Smooth-On

Marine Collagen: Amazon

Bentonite: Soap-and-More

Hemp: Fiber samples were supplied from Dr. Jan Slaski (Principal researcher, InnoTech Alberta in Vegreville, Alberta).

Task 2: Smooth-On

Wood flour: Industrial Plastics & Paints

The embodiments described herein are intended to be examples only.

Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill of those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1-48. (canceled)

49. A synthetic bone comprising:
a plastic casting resin,
a pore-inducing additive, and
a hardness-altering additive.

50. The synthetic bone of claim 49, wherein the plastic casting resin is Smooth-Cast 300, Smooth-Cast 321, MirrorCast, Smooth-Cast 385, Smooth-Cast 380, Smooth-Cast ONYX, Task 15, Task 2, Task 11, or Smooth-Cast 60D.

51. The synthetic bone of claim 49, wherein the plastic casting resin is selected from Smooth-Cast 300 or Task 2.

52. The synthetic bone of claim 49, wherein the pore-inducing additive is bentonite, wood ash, fly ash, volcanic ash, soda ash, kaolinite, talc, calcium carbonate or montmorillonite.

53. The synthetic bone of claim 49, wherein the pore-inducing additive is calcium carbonate or bentonite.

54. The synthetic bone of claim 49, wherein the hardness-altering additive is collagen AquaResin powder, Goldfinger Powder, Ure-Fil 7, QuarryTone, Ure-Fil 3, Ure-Fil 15, gelatin, Ure-Fil 11, or wood flour.

55. The synthetic bone of claim 49, wherein the hardness-altering additive is collagen or wood flour.

56. The synthetic bone of claim 49, the bone further comprising a fiber.

57. The synthetic bone of claim 56, wherein the fiber is hemp, jute, flax, ramie, sisal, bagasse, wood fibers, bamboo fibers, wood shavings, bleached or unbleached kraft, animal fibers, silk, wool, semi-synthetic fiber, nylon, Dacron, rayon, acrylic polyesters, cotton, tencel lyocell, linen (flax), or alkali resistant glass fibers.

58. The synthetic bone of claim 49, wherein the plastic casting resin is Smooth-Cast 300, the pore-inducing additive is bentonite, and the hardness-altering additive is collagen, wherein the bone further comprises a fiber and the fiber is hemp.

59. The synthetic bone of claim 49, wherein the plastic casting resin is Task 2, the pore-inducing additive is calcium carbonate, and the hardness-altering additive is wood flour.

60. A synthetic bone, comprising:
a plastic casting resin,
a pore-inducing additive, and
a softening additive.

61. The synthetic bone of claim 60, wherein the plastic casting resin is Smooth-Cast 300, Smooth-Cast 321, MirrorCast, Smooth-Cast 385, Smooth-Cast 380, Smooth-Cast ONYX, Task 15, Task 2, Task 11, or Smooth-Cast 60D.

62. The synthetic bone of claim 60, wherein the plastic casting resin is Task 2.

63. The synthetic bone of claim 60, wherein the pore-inducing additive is calcium carbonate, bentonite, wood ash, fly ash, volcanic ash, soda ash, kaolinite, talc, or montmorillonite.

64. The synthetic bone of claim 60, wherein the pore-inducing additive is calcium carbonate, bentonite, or a combination thereof.

65. The synthetic bone of claim 60, wherein the softening additive is wood flour or collagen.

66. The synthetic bone of claim 60, wherein the plastic casting resin is Task 2, the pore-inducing additive is calcium carbonate, and the softening additive is wood flour.

67. The synthetic bone of claim 60, wherein the plastic casting resin is Task 2, the pore-inducing additive is calcium carbonate and bentonite, and the softening additive is wood flour.

68. The synthetic bone of claim 60, further comprising a fat-simulating substance.

69. The synthetic bone of claim 68, wherein the fat-simulating substance is petroleum jelly, glycerin, soft paraffin and/or mineral oil.

70. The synthetic bone of claim 60 forming an inner bone core layer and further comprising an outer synthetic bone layer comprising:
a plastic casting resin,
a pore-inducing additive, and
a hardness-altering additive, wherein the hardness-altering additive is collagen, AquaResin powder, Goldfinger Powder, Ure-Fil 7, QuarryTone, Ure-Fil 3, Ure-Fil 15, gelatin, Ure-Fil 11, or wood flour.
